(19) United States
(12) Patent Application Publication
(10) Pub. No.: US 2024/0366500 A1
(43) Pub. Date: Nov. 7, 2024

(54) INJECTABLE COMPOSITIONS AND RELATED METHODS

(71) Applicant: Boston Scientific Scimed, Inc., Maple Grove, MN (US)

(72) Inventors: Siavash Sam PARKHIDEH, Marlborough, MA (US); Lauren Sfakis LYDECKER, Millbury, MA (US)

(73) Assignee: Boston Scientific Scimed, Inc., Maple Grove, MN (US)

(21) Appl. No.: 18/650,661

(22) Filed: Apr. 30, 2024

Related U.S. Application Data
(60) Provisional application No. 63/500,080, filed on May 4, 2023.

(51) Int. Cl. A61K 9/00; A61K 9/06; A61K 47/02; A61K 47/10; A61K 47/38; A61M 5/32; A61P 41/00

(52) U.S. Cl. CPC A61K 9/0024; A61K 9/06; A61K 47/02; A61K 47/10; A61K 47/38; A61M 5/32; A61P 41/00

(57) ABSTRACT

Injectable compositions and methods of use are discussed. The injectable composition may include at least one biocompatible material chosen from polyethylene glycol (PEG), alginate, methylcellulose, carboxymethylcellulose, hydroxypropylmethylcellulose, chitosan, carboxymethylchitosan, laponite, gelatin, xanthan gum, or a combination thereof, and a biocompatible salt solution. The composition may be formulated to form a gel in submucosal tissue to separate tissue layers. The viscosity of the composition may decrease by at least 50% at 37° C. within 24 hours.

### CROSS-REFERENCE TO RELATED APPLICATIONS

This application claims the benefit of priority to U.S. Provisional Application No. 63/500,080, filed on May 4, 2023, which is incorporated by reference herein in its entirety.

### TECHNICAL FIELD

Various aspects of this disclosure relate generally to compositions and related methods useful for medical procedures. In particular, injectable compositions for separating tissue layers during, e.g., endoscopic procedures.

### BACKGROUND

Endoscopic mucosal resection (EMR) and endoscopic submucosal dissection (ESD) for removal of a lesion typically use a lifting agent to separate the gastrointestinal (GI) tract mucosa layer (and lesion) from the GI tract muscular layer, and reduce the risk of GI tract perforation. While saline is commonly used as a lifting agent, it is rapidly absorbed by the body and typically requires multiple reinjections to perform a prolonged medical procedure.

### SUMMARY

The present disclosure includes compositions and methods useful for separating tissue layers during, e.g., endoscopic procedures. According to some aspects of the present disclosure, the composition may include at least one biocompatible material chosen from polyethylene glycol (PEG), alginate, methylcellulose, carboxymethylcellulose, hydroxypropylmethylcellulose, chitosan, carboxymethylchitosan, laponite, gelatin, xanthan gum, or a combination thereof, and a biocompatible salt solution, wherein the composition is formulated to form a gel in submucosal tissue to separate tissue layers, and wherein a viscosity of the composition decreases by at least 50% at 37° C. within 24 hours.

According to some aspects, the composition may include about 0.125% w/v to about 3% w/v PEG. In some examples, the molecular weight of the PEG may range from about 400 Da to 8000 Da. In some examples, the molecular weight of the PEG may range from about 1000 Da to 4000 Da. In some examples, the composition may include from about 1% w/v to about 2% w/v PEG. In some examples, the molecular weight of the PEG may be about 3350 Da. In some examples, the biocompatible salt may include sodium chloride of saline solution, and the saline solution has a concentration of about 0.7% w/v to about 1.2% w/v. In some examples, the composition may include from about 0.01% w/v to about 0.6% w/v alginate. In some examples, the composition may include from about 0.1% w/v to about 0.4% w/v methylcellulose. In some examples, the composition may include from about 0.1% w/v to about 0.6% w/v hydroxypropylmethylcellulose. In some examples, the composition may include from about 0.1% w/v to about 0.4% w/v chitosan.

According to some aspects, a syringe may include the composition. In some examples, the syringe may include a 23 gauge needle, and the composition may have an injection force through the needle less than 60 N.

The present disclosure includes, for example, a method of treating a subject. In some examples, the method may include injecting the composition into submucosal tissue of the subject. In some examples, the composition may provide a lifting force of at least 6 mm for at least 90 minutes after injection.

The present disclosure includes, for example, a composition including about 0.125% w/v to about 3% w/v of a polyethylene glycol (PEG) polymer having a molecular weight ranging from about 400 Da to about 8000 Da, a biocompatible salt solution, wherein the composition is formulated to form a gel in submucosal tissue to separate tissue layers.

According to some aspects, a viscosity of the composition may decrease by at least 50% at 37° C. within 24 hours. In some examples, the PEG polymer may have a molecular weight of about 3500 Da.

The present disclosure includes, for example, a method of treating a subject including injecting a composition into submucosal tissue of the subject, wherein the composition may include about 0.125% w/v to about 3% w/v of a polyethylene glycol (PEG) polymer, and a biocompatible salt solution, wherein the composition may form a gel in the submucosal tissue to separate tissue layers, and wherein the PEG polymer may have a molecular weight ranging from about 400 Da to about 8000 Da. In some examples, the composition may provide a lifting force of at least 8 mm for at least 60 minutes after injection.

### BRIEF DESCRIPTION OF THE FIGURES

The accompanying drawings, which are incorporated in and constitute a part of this application, illustrate aspects of the present disclosure and together with the description, serve to explain the principles of the present disclosure.

FIG. 1 shows injection force as a function of syringe plunger distance, as discussed in Example 1.

FIG. 2 shows tissue lift height as a function of time, as discussed in Example 2.

FIG. 3 shows viscosity change as a function of time, as discussed in Example 3.

FIGS. 4A-4C show tissue lift height as a function of time and corresponding photographs, as discussed in Example 5.

FIGS. 5A and 5B show normalized tissue lift height as a function of time and a corresponding photograph, as discussed in Example 6.

FIGS. 6A and 6B show normalized tissue lift height as a function of time and a corresponding photograph, as discussed in Example 7.

FIGS. 7A and 7B show injection force data, as discussed in Example 8.

### DETAILED DESCRIPTION

Both the foregoing general description and the following detailed description are exemplary and explanatory only and are not restrictive of the features, as claimed.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass±5% of a specified amount or value. All ranges are understood to include endpoints, e.g., an amount ranging from 0.1 g to 10 g includes 0.1 g, 10 g, and all values between.

The present disclosure includes injectable compositions formulated to form a gel, e.g., for separating tissue layers during medical procedures, e.g., endoscopic procedures. The compositions may be injected into the GI tract submucosal layer to lift and/or raise the GI tract mucosa layer and/or lesion away from the GI tract muscular layer during, e.g. EMR or ESD. The compositions may reduce the risk of tissue perforation of the GI tract. The compositions may be biodegradable. The compositions herein may be delivered to a subject by a suitable medical device such as a catheter inserted through an endoscope.

The compositions herein may offer a range of properties and/or characteristics, including among others, decreasing viscosity under shear strain (shear-thinning). With such properties, the composition may be injected and delivered to target sites via suitable medical devices such as, e.g., single/multi lumen catheters, including endoscopes, and syringes, among other devices useful for medical procedures. The compositions herein may form a gel at a target site and/or separate layers of tissue. The compositions herein may separate tissue for an extended period of time and may reduce and/or eliminate the need for multiple injections during a medical procedure, e.g., during EMR or ESD. The compositions herein may be biocompatible.

The compositions herein may include one or more biocompatible materials and a physiological salt solution. For example, the one or more biocompatible materials may comprise polyethylene glycol (PEG), alginate, carboxymethylcellulose, chitosan (including, e.g., carboxymethylchitosan), methylcellulose, hydroxypropylmethylcellulose, laponite, gelatin, xanthan gum, and combinations thereof. The biocompatible material may comprise a mixture of nanosilicates, gelatin and tantalum, e.g., gelatin and nanoclay mixtures.

For polymers like PEG that may have different molecular weight or encompass a range of molecular weights depending on the amount of polymerization or crosslinking, the biocompatible material(s) may be selected to have a molecular weight suitable for the desired properties of the composition. For example, greater polymerization and crosslinking may provide for materials with longer degradation times as compared to materials with less polymerization and crosslinking. Further, for example, the molecular weight of the biocompatible material may affect other properties of the composition such as viscosity or shear strength.

According to some aspects of the present disclosure, the biocompatible material(s) may comprise PEG having a molecular weight ranging from about 400 Da (g/mol) to about 8000 Da, such as from about 400 Da to about 600 Da, about 900 Da to about 1000 Da, about 1450 Da to about 1540 Da, about 2000 Da to about 3000 Da, about 3350 Da to about 4000 Da, or about 4600 Da to about 8000 Da, among other possibilities. In some examples, the biocompatible material(s) comprises hydroxypropylmethylcellulose having a molecular weight less than 50,000 Da, e.g., within a range of about 10,000 Da to 50,000 Da. In some examples, the biocompatible material(s) comprises hydroxypropylmethylcellulose having a molecular weight greater than or equal to 50,000 Da, such as ranging from about 50,000 Da to 60,000 Da. In some examples, the biocompatible material(s) comprises chitosan having a molecular weight less than 100,000 Da, e.g., ranging from about 25,000 Da to 100,000 Da.

According to some aspects of the present disclosure, the composition may comprise from about 0.01% w/v to about 30% w/v of the biocompatible material(s), such as from about 5% w/v to about 15% w/v, from about 2% w/v to about 7% w/v, from about 12% w/v to about 20% w/v, from about 8% w/v to about 16% w/v, from about 4% w/v to about 10% w/v, from about 0.1% w/v to about 8% w/v, from about 0.125% w/v to about 3.0% w/v, from about 1.0% w/v to about 2.0% w/v, from about 0.01% w/v to about 0.6% w/v, from about 0.1% w/v to about 0.6% w/v, or from about 0.1% w/v to about 0.4% w/v.

In some examples, the composition comprises from about 0.125% w/v to about 20.0% w/v of PEG, such as from about 0.0125% w/v to about 3% w/v of PEG, or from about 1.0% w/v to about 2.0% w/v of PEG. In some examples, the composition comprises at least 12.5% w/v of PEG, such as ranging from about 12.5% w/v to about 20% w/v of PEG. In some examples, the composition comprises from about 0.01% w/v to about 0.6% w/v alginate, such as about 0.0625% w/v, about 0.125% w/v, about 0.25% w/v, or about 0.5% w/v. In some examples, the composition comprises from about 0.1% w/v to about 0.6% w/v hydroxypropylmethylcellulose, such as about 0.125% w/v, about 0.25% w/v, or about 0.5% w/v. In some examples, the composition comprises from about 0.1% w/v to about 0.4% w/v methylcellulose, such as about 0.125% w/v or about 0.25% w/v. In some examples, the composition comprises from about 0.1% w/v to about 0.6% w/v chitosan, such as about 0.25% w/v or about 0.5% w/v.

The compositions may comprise a biocompatible and/or physiological salt solution, for example, a saline solution of sodium chloride. In some examples, the composition may comprise saline solution having a concentration ranging from about 0.7% w/v to about 1.2% w/v, e.g., about 0.9% w/v.

As discussed above, the compositions herein may be delivered to a target site of a subject via a suitable medical device. For example, the compositions herein may be loaded into a syringe coupled to an injection needle, and injected into submucosal tissue. The compositions herein may form a gel in the submucosal tissue to separate tissue layers. The compositions may have shear strength properties to facilitate injection. For example, when passed through a 23 gauge needle, the composition may exhibit an injection force less than 60 N, such as ranging from about 20 N to 60 N or ranging from about 20 N to about 50 N. In some examples, the compositions herein may exhibit an injection force through a 23 gauge needle less than 50 N, such as ranging from about 30 N to 50 N.

The compositions herein may provide adequate tissue layer separation and/or distance between tissue layers during a medical procedure. In some examples, the distance between tissue layers provided by the composition upon injection may be at least 6 mm, e.g., ranging from 6 mm to 15 mm. For example, the distance between tissue layers may be at least 8 mm, at least 10 mm, or at least 12 mm. The distance between tissue layers may be maintained for at least 30 minutes, e.g., from 30 minutes to about 2 hours, or about 90 minutes. In some examples herein, the distance between tissue layers maintained by the composition may be at least 8 mm for at least 60 minutes. In some examples, the distance between tissue layers maintained by the composition may be at least 10 mm for at least 30 minutes. In some examples, the distance between tissue layers maintained by the composition may be at least 12 mm during initial injection.

The viscosity of the compositions herein may decrease over time at 37° C., e.g., during degradation or bioresorption. For example, the viscosity may decrease by at least 50% within 24 hours at 37° C. after injection. In some examples, the viscosity may decrease by at least 70% within 72 hours 37° C. after injection.

### EXAMPLES

The following examples are intended to illustrate aspects of the present disclosure without, however, being limiting in nature. It is understood that the present disclosure encompasses additional embodiments consistent with the foregoing description and following examples. The present disclosure is not limited to the examples further described below and encompasses additional conditions without departing from the scope of the present disclosure.

### Example 1

Tests were conducted to assess injection force of a composition comprising PEG as compared to saline solution (NERL™ blood bank saline). PEG 3350 (Sigma Aldrich) was dissolved at 2% w/v in 0.9% w/v saline solution. One drop of FD&C Blue 1 was used in every 20 mL of solution to improve visualization. Sterile solutions were prepared by bottle-top vacuum filtration through 0.2 μm aPES filter membranes.

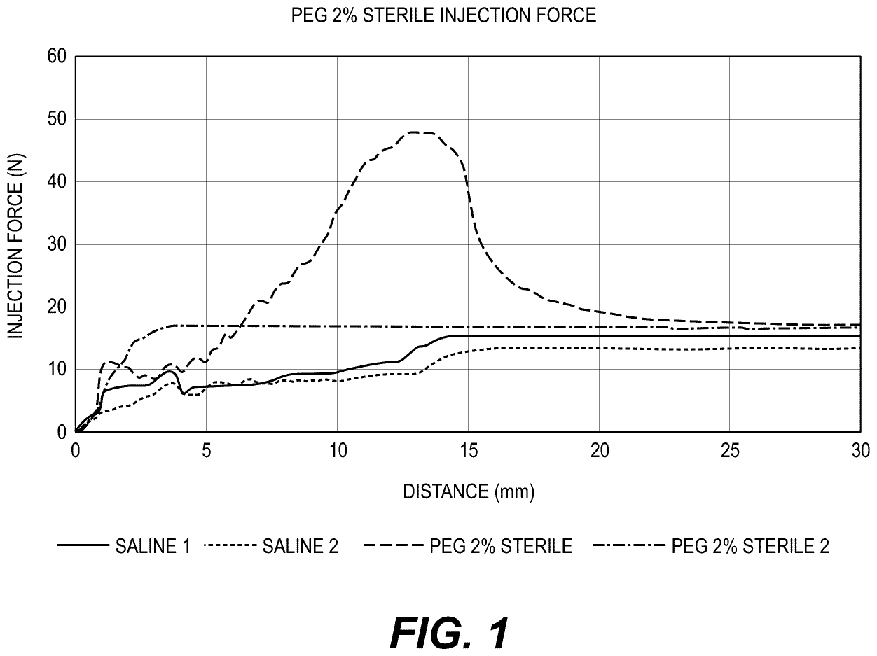

Injection force was measured for two samples of each solution. Each sample was loaded into a glass syringe with a 23 gauge needle. Solutions were injected at 0.4 mL/s and injection force measured as the syringe plunger moved a distance of 30 mm. FIG. 1 shows injection force (N) as a function of syringe plunger distance (mm). The PEG 3350 solution exhibited an injection force of less than 60 N maximum force.

### Example 2

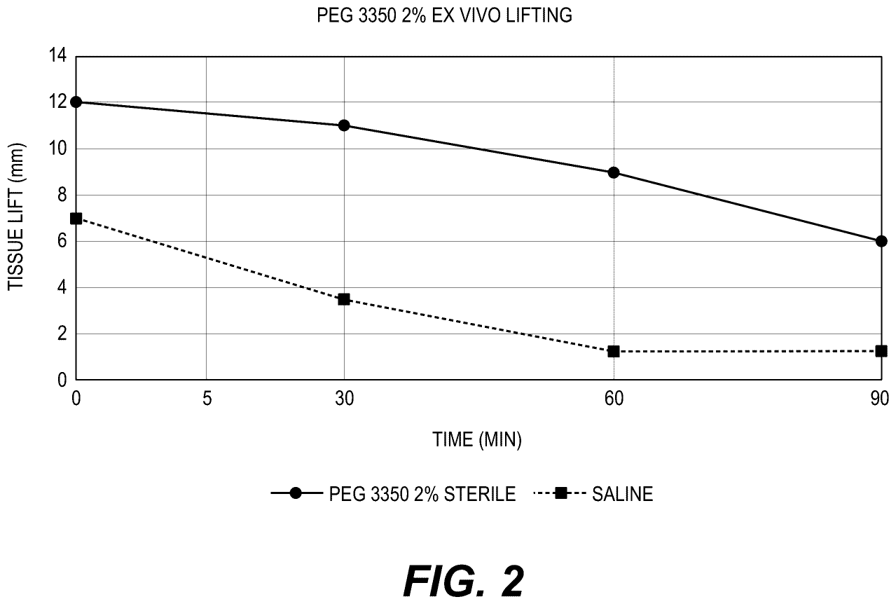

The ability for the compositions of Example 1 to lift tissue was evaluated in an ex vivo model. Two cc of the PEG 3350 solution was injected into the submucosa of porcine colon tissue with a 10 mL syringe and 23 gauge needle. Height of the tissue was measured every 30 minutes. FIG. 2 shows tissue lift height (mm) as a function of time (min). For the PEG 3350 solution, the initial injection provided a height of about 12 mm (0 minutes), decreasing to about 11 mm at 30 minutes, about 9 mm at 60 minutes, and about 6 mm at 90 minutes. The saline solution provided markedly different behavior, quickly losing height.

### Example 3

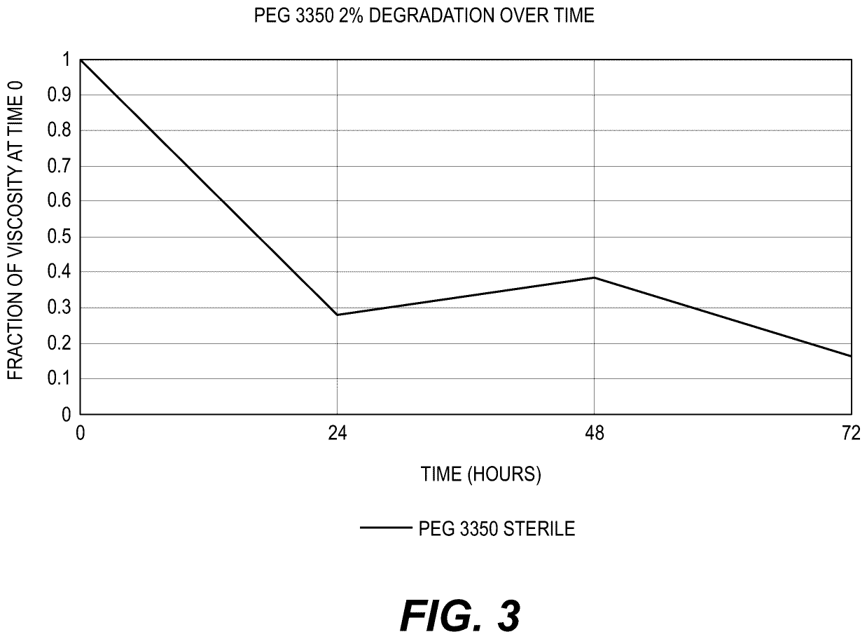

Degradation of the PEG 3350 solution of Examples 1 and 2 was investigated to assess how the material may behave in the body after injection. Viscosity change was monitored over time at 37° C. As shown in FIG. 3, viscosity decreased by approximately 70% at 24 hours.

### Example 4

Various compositions (A-S) were prepared according to Tables 1-7 below. Comparative compositions (T-V) were prepared according to Tables 8 and 9. Compositions A-Q and T-V were prepared in 0.9% saline solution. Compositions R and S were prepared in water. The injection force tested as described in Example 2 is also reported. Compositions A-S exhibited an injection force within target specifications, whereas Compositions T-V exhibited an injection force outside desired ranges.

TABLE 1

| | PEG molecular | PEG | Injection |
|---|---|---|---|
| Composition | weight (Da) | (% w/v) | Force (N) |
| A | 4000 | 2 | 33 |
| B | 400 | 2 | 15.92 |
| C | 1000 | 2 | 16.3 |
| D | 4000 | 2 | 21.76 |
| E | 8000 | 2 | 20.06 |

TABLE 2

| Composition | Laponite (% w/v) | Injection Force (N) |
|---|---|---|
| F | 2 | 48.39 |

TABLE 3

| Composition | Alginate (% w/v) | Injection Force (N) |
|---|---|---|
| G | 0.0625 | 20.29 |
| H | 0.125 | 17.75 |
| I | 0.25 | 24.59 |
| J | 0.5 | 34.61 |

TABLE 4

| Composition | Methylcellulose (% w/v) | Injection Force (N) |
|---|---|---|
| K | 0.125 | 46.51 |
| L | 0.25 | 46.51 |

TABLE 5

| | Hydroxypropylmethylcellulose | Injection |
|---|---|---|
| Composition | (% w/v) | Force (N) |
| M | 0.5 | 48.37 |
| N | 0.125 | 34.95 |
| O | 0.25 | 59.76 |

TABLE 6

| Composition | Chitosan (% w/v) | Injection Force (N) |
|---|---|---|
| P | 0.25 | 29.09 |
| Q | 0.5 | 50.50 |

TABLE 7

| Composition | Laponite (% w/v) | Gelatin (% w/v) | Injection Force (N) |
|---|---|---|---|
| R | 1.114 | 1.080 | 29.00 |
| S | 2.228 | 1.080 | 63.00 |

TABLE 8

| Composition | Alginate (% w/v) | Injection Force (N) |
|---|---|---|
| T | 1 | 294 |
| U | 2 | 499 |

TABLE 9

| Composition | Chitosan (% w/v) | Injection Force (N) |
|---|---|---|
| V | 2.0 | 559 |

### Example 5

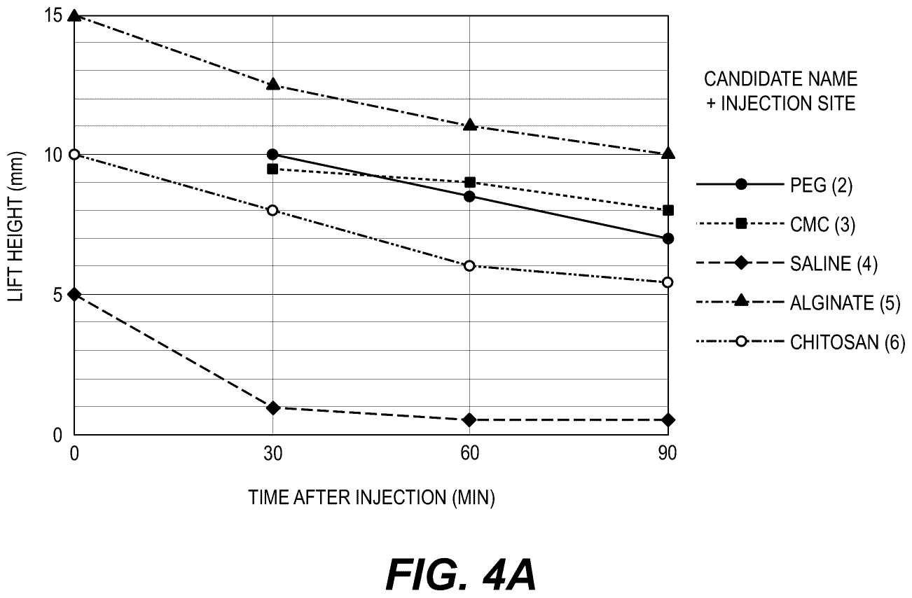

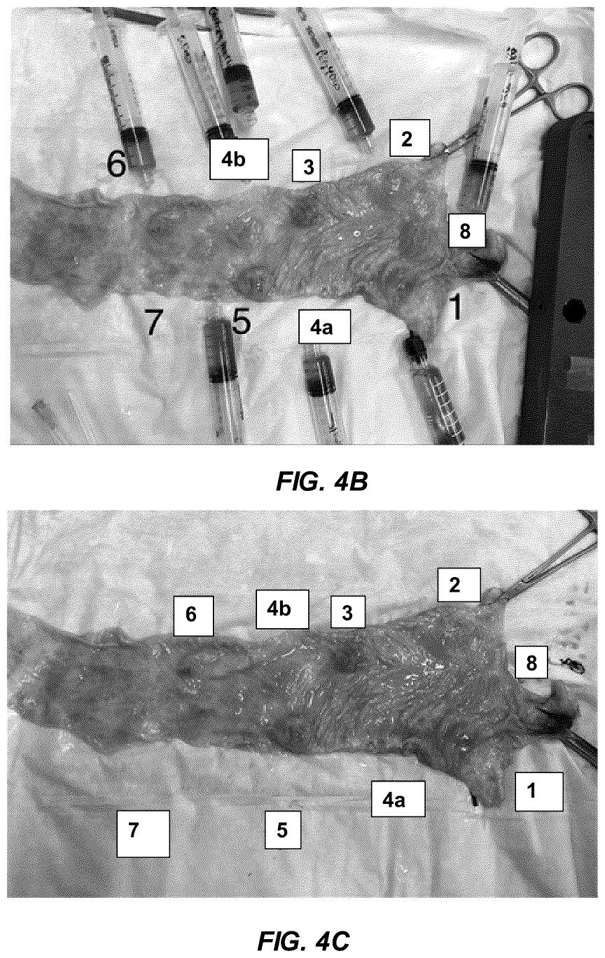

Compositions were prepared with PEG 4000 2% w/v (2), carboxymethylcellulose (CMVC) 2% w/v (3), saline 0.9% w/v (4), alginate 2% w/v (5), and chitosan 2% w/v (6) to evaluate the ability to lift tissue in an ex vivo model. Porcine colon tissue was thawed overnight. Two cc of each composition was injected into submucosa of sigmoid and descending colon with a 10 mL Luer lock syringe and 25 gauge needle. Heights of the tissue were measured every 30 minutes with a ruler. FIG. 4A shows tissue lift height (mm) as a function of time (min). FIG. 4B is a photograph of the porcine colon tissue at initial injection of the compositions at different locations and FIG. 4C is a photograph of the porcine colon 60 to 90 minutes after injection.

### Example 6

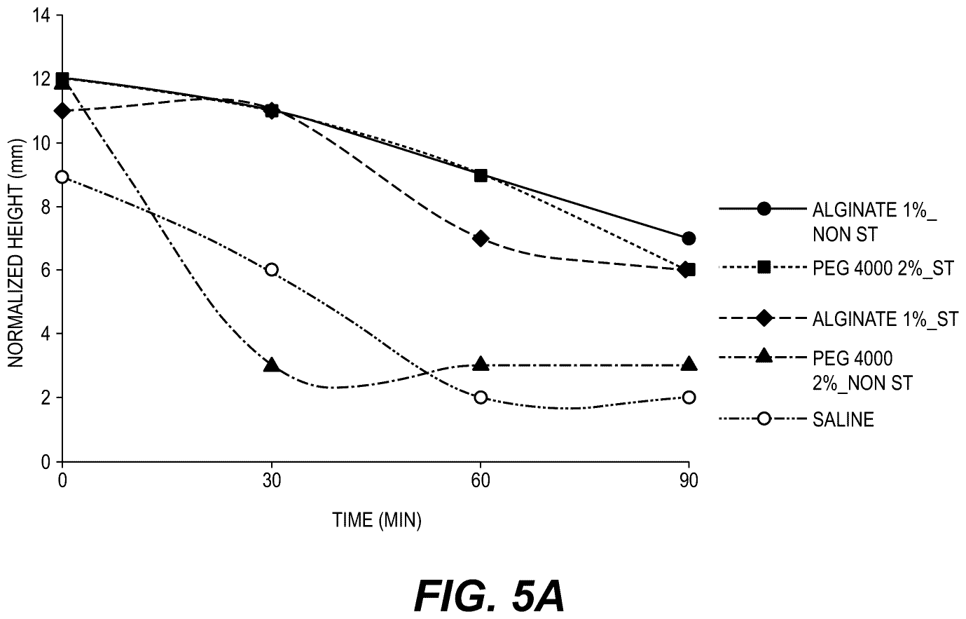

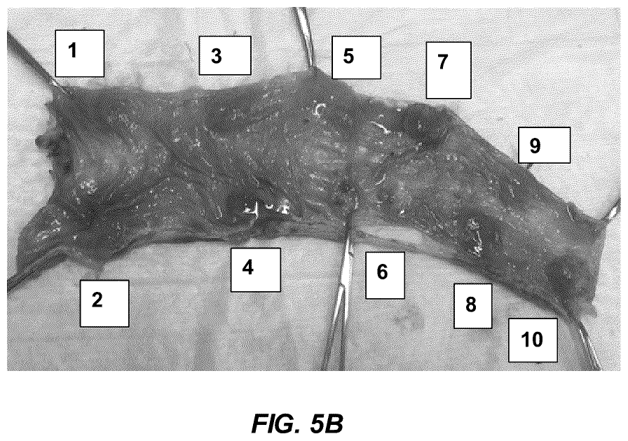

Compositions were prepared with alginate 1% w/v (non-sterile), PEG 4000 2% w/v (non-sterile), alginate 1% w/v (sterile), PEG 4000 2% w/v (sterile), and saline 0.9% w/v to evaluate the ability to lift tissue in an ex vivo model. FD&C Blue 1 was used in each composition to improve visualization. Two cc of each composition was injected into the submucosa of female porcine tissue with a 10 mL syringe and 25 gauge needle. FIG. 5A shows normalized tissue height (mm) as a function of time (min), and FIG. 5B is a photograph of the porcine colon tissue after injection of the compositions at different locations in the tissue. In FIG. 5B, sample 2 corresponds to PEG 4000 2% w/v (sterile), sample 3 corresponds to alginate 1% w/v (non-sterile), sample 9 corresponds to saline 0.9% w/v, and sample 10 corresponds to alginate 1% w/v (sterile).

### Example 7

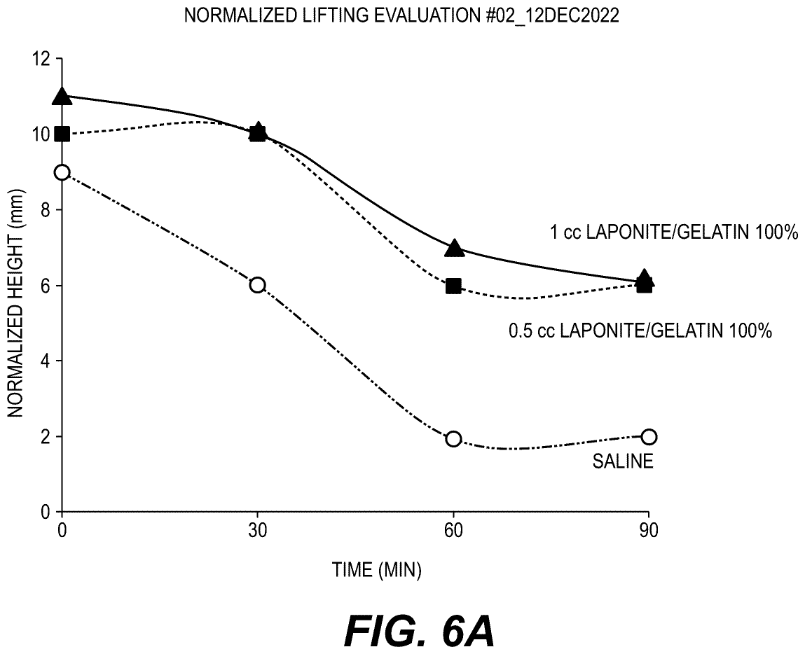

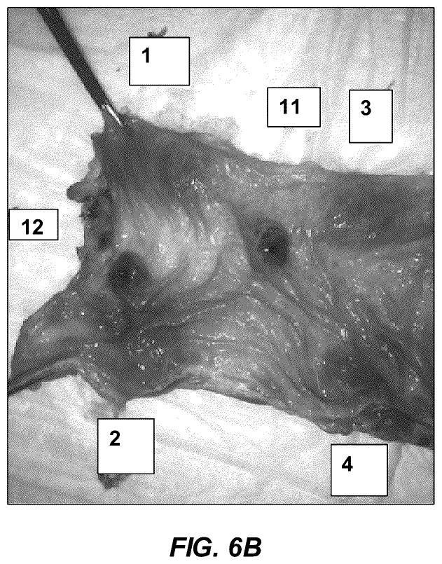

Compositions were prepared with laponite 8.910% w/v/gelatin 1.080% w/v (non-sterile) and saline 0.9% w/v to evaluate the ability to lift tissue in an ex vivo model. A sample of 0.5 mL or 1 mL was injected into the submucosa of porcine colon tissue. FIG. 6A shows normalized tissue height (mm) as a function of time (min), and FIG. 6B is a photograph of the porcine colon tissue after injection of the compositions at different locations in the tissue. In FIG. 6B, sample 11 corresponds to a 1 mL injection of laponite 8.910% w/v/gelatin 1.080% w/v (non-sterile) and sample 12 corresponds to a 0.5 mL injection of laponite 8.910% w/v/gelatin 1.080% w/v (non-sterile).

### Example 8

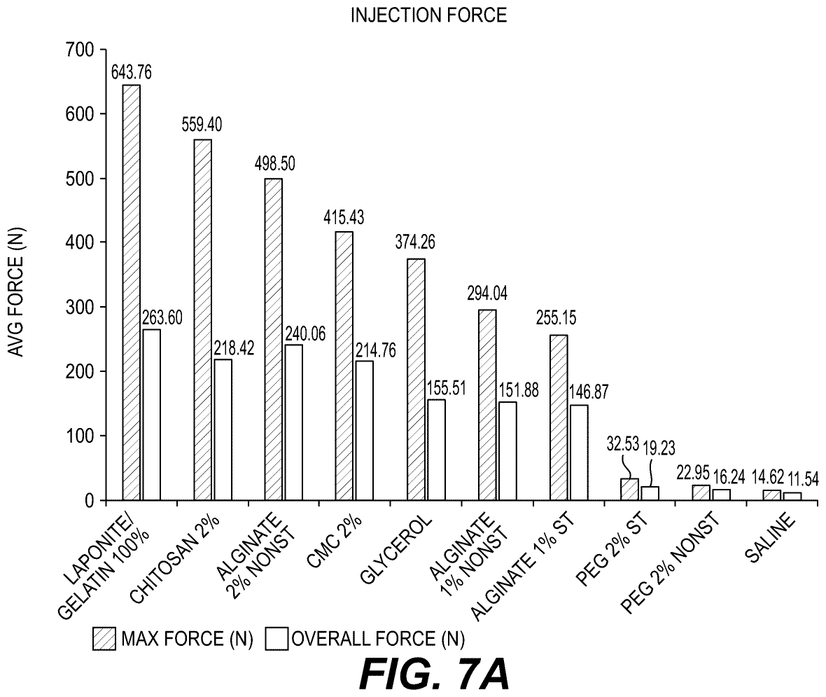

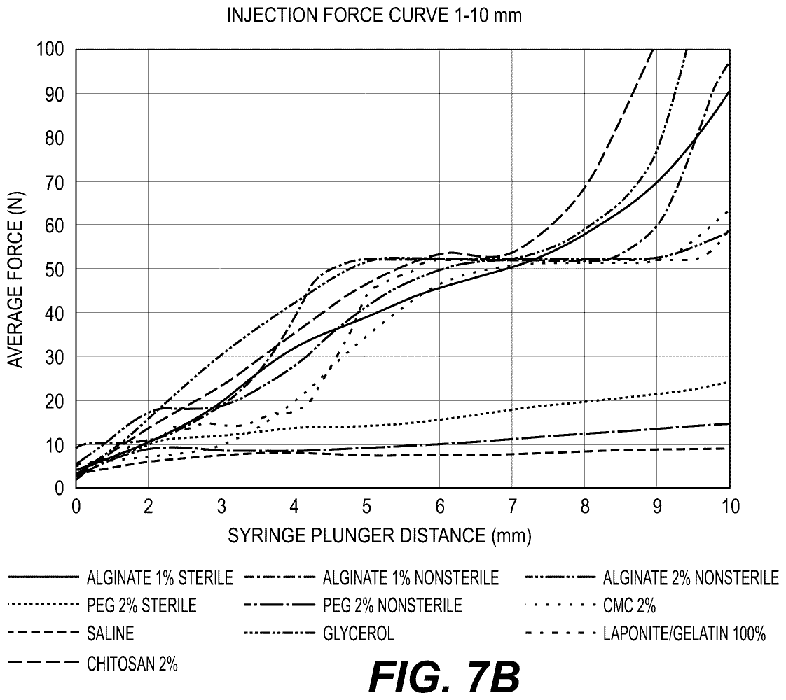

As discussed above in Example 4, injection force was measured for various compositions prepared with laponite 8.910% w/v/gelatin 1.080% w/v, chitosan 2% w/v, alginate 2% w/v (non-sterile), carboxymethylcellulose (CMC) 2% w/v, glycerol, alginate 1% w/v (non-sterile), alginate 1% w/v (sterile), PEG 4000 2% w/v (sterile), PEG 4000 2% w/v (non-sterile), and saline 0.9% w/v. FIG. 7A shows maximum injection force (N) and overall injection force (N) for each composition. FIG. 7B also shows average force (N) as a function of syringe plunger distance (mm). The composition prepared with laponite 8.910% w/v/gelatin 1.080% w/v was observed to have the highest maximum force.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only, with a true scope and spirit of the present disclosure being indicated by the following claims.

We claim:

1. An injectable composition comprising:
at least one biocompatible material chosen from polyethylene glycol (PEG), alginate, methylcellulose, carboxymethylcellulose, hydroxypropylmethylcellulose, chitosan, carboxymethylchitosan, laponite, gelatin, xanthan gum, or a combination thereof; and
a biocompatible salt solution;
wherein the composition is formulated to form a gel in submucosal tissue to separate tissue layers; and
wherein a viscosity of the composition decreases by at least 50% at 37° C. within 24 hours.

2. The composition of claim 1, wherein the composition comprises from about 0.125% w/v to about 3% w/v PEG.

3. The composition of claim 2, wherein the composition comprises from about 1% w/v to about 2% w/v PEG.

4. The composition of claim 1, wherein the composition comprises PEG and the molecular weight of the PEG ranges from about 400 Da to 8000 Da.

5. The composition of claim 4, wherein a molecular weight of the PEG ranges from about 1000 Da to 4000 Da.

6. The composition of claim 4, wherein the molecular weight of the PEG is about 3350 Da.

7. The composition of claim 1, wherein the biocompatible salt comprises sodium chloride of saline solution, and the saline solution has a concentration of about 0.7% w/v to about 1.2% w/v.

8. The composition of claim 1, wherein the composition comprises from about 0.01% w/v to about 0.6% w/v alginate.

9. The composition of claim 1, wherein the composition comprises from about 0.1% w/v to about 0.4% w/v methylcellulose.

10. The composition of claim 1, wherein the composition comprises from about 0.1% w/v to about 0.6% w/v hydroxypropylmethylcellulose.

11. The composition of claim 1, wherein the composition comprises from about 0.1% w/v to about 0.4% w/v chitosan.

12. A syringe comprising the composition of claim 1.

13. The syringe of claim 12, wherein the syringe includes a 23 gauge needle, and the composition has an injection force through the needle less than 60 N.

14. A method of treating a subject, the method comprising injecting the composition of claim 1 into submucosal tissue of the subject.

15. The method of claim 14, wherein the composition provides a lifting force of at least 6 mm for at least 90 minutes after injection.

16. An injectable composition comprising:
about 0.125% w/v to about 3% w/v of a polyethylene glycol (PEG) polymer having a molecular weight ranging from about 400 Da to about 8000 Da; and
a biocompatible salt solution;
wherein the composition is formulated to form a gel in submucosal tissue to separate tissue layers.

17. The composition of claim 16, wherein a viscosity of the composition decreases by at least 50% at 37° C. within 24 hours.

18. The composition of claim 16, wherein the PEG polymer has a molecular weight of about 3500 Da.

19. A method of treating a subject comprising injecting a composition into submucosal tissue of the subject, wherein the composition comprises:
about 0.125% w/v to about 3% w/v of a polyethylene glycol (PEG) polymer; and
a biocompatible salt solution,
wherein the composition forms a gel in the submucosal tissue to separate tissue layers, and
wherein the PEG polymer has a molecular weight ranging from about 400 Da to about 8000 Da.

20. The method of claim 19, wherein the composition provides a lifting force of at least 8 mm for at least 60 minutes after injection.
